(19) United States
(12) Patent Application Publication
(10) Pub. No.: US 2005/0283187 A1
(43) Pub. Date: Dec. 22, 2005

(54) Vascular occlusion device

(76) Inventor: Matthew S. Longson, Holliday, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/157,258

(22) Filed: Jun. 21, 2005

Related U.S. Application Data
(60) Provisional application No. 60/581,947, filed on Jun. 22, 2004.

(51) Int. Cl. A61B 17/08

(52) U.S. Cl. 606/213

(57) ABSTRACT

The present invention relates to sealing an access site within a subcutaneous tissue structure with an occlusion device comprising submucosal tissue. The occlusion device a stem and a flexible head, where the head is a concave dome, and has one or more ribs which strengthen the head and reduce flexure that would invert the dome. The stem comprises a loop configured to receive a suture which may be stitched to the patient and whereby the occlusion device is secured. The occlusion device comprises a transition section which is tapered and which connects the head to the stem. The tapered section is configured to strengthen the occlusion device and to facilitate positioning of the device within the subcutaneous tissue structure. A delivery device is configured to releasably secure stem the occlusion device and facilitate delivery of the occlusion device to a vascular access site, and to facilitate bending of the head.

### CROSS-REFERENCE TO RELATED APPLICATIONS

This application claims the benefit of U.S. Provisional Patent Application No. 60/581,947, filed Jun. 22, 2004, which is incorporated herein by reference.

### BACKGROUND OF THE INVENTION

1. The Field of the Invention

Exemplary embodiments of the invention relate to the field of sealing access sites in blood vessels, body cavities, and bodily organs. More particularly, the invention relates to apparatus, methods, and kits for occluding a vascular access site.

2. The Relevant Technology

An important element in any medical procedure is the control and stoppage of blood loss. Stopping blood loss is a particular concern in intravascular medical procedures where a laceration in a vein or artery is made to grant venous or arterial access. Commonly, these procedures include the insertion, use, and removal of a catheter device to diagnose or repair a medical condition. Intravascular procedures of this type represent a significant number of the medical procedures performed each year—well into the hundreds of thousands—thus providing at least an equal number of procedures where excessive bleeding is a concern.

Accessing a vein or artery typically requires entrance through a wall of the blood vessel, which further requires that an access site be selected and the vessel wall be lacerated or punctured. This access site is of particular concern. If the site is left unsealed, blood may escape and enter into the surrounding body cavities and tissue. Where excessive blood escapes, the effectiveness of the medical procedure may be compromised and complications may arise. To avoid or counter these complications, the medical staff must be vigilant in providing continued care to the patient following an intravascular procedure.

One method used to avoid excessive bleeding is to apply pressure to the affected area. This process attempts to block flow from the vein or artery until the natural clotting process is complete. Pressure may be manually applied, or with a sandbag, bandage, or clamp. The effectiveness of this pressure is compromised unless the patient remains motionless while pressure is applied. Patients are monitored during the time during which clotting is occurring, and this natural process generally takes at least one to two hours, and sometimes patients must remain motionless and be monitored for up to three to nine hours. The need to remain motionless makes patients uncomfortable and increases the length of the hospital stay and the corresponding costs of medical care.

Additional techniques have been developed to reduce the amount of time for hemostasis. Alternative techniques may include some type of plug or barrier that occludes the laceration in the blood vessel wall. For example, collagen plugs are well known in the art. When a collagen plug is inserted, the blood or other body fluids cause the collagen plug to swell, such that it blocks the access site. A concern with the use of any plug is that the plug may block the flow of blood in the vessel, or that the. plug may be released into the blood stream where it can float into a smaller vessel and embolize.

Additional concerns with existing plug techniques are the need for specialized equipment, the time required for installation, and the complexity of the procedure. Sealing the access site generally requires that standard equipment be abandoned, and specialized equipment and technology be used to facilitate insertion and positioning of the plug. Alternatively, other techniques may be adaptable to the use of traditional, stock equipment, but require additional steps to install the plug. These additional steps take additional time which results in a greater cost to the patient, as well as resulting in an increase in the blood loss during the time between completion of the procedure and hemostasis.

### BRIEF SUMMARY OF THE INVENTION

Exemplary embodiments of the invention relate to a vascular occlusion device that substantially seals a subcutaneous tissue structure such as a blood vessel following a percutaneous medical procedure. The vascular occlusion device reduces the risk of bleeding following a medical procedure by improving the ability of medical personnel to quickly and easily seal an access site of a blood vessel. The use of the vascular occlusion device accelerates hemostasis in the patient, thus reducing the health risks associated with excess blood loss. Additionally, the vascular occlusion device allows a patient a near full range of motion soon after surgery, thus reducing the expenses of the procedure and corresponding hospital stay.

In one exemplary embodiment, the vascular occlusion device comprises a stem that is directly connected to a head. In this embodiment, the head is concave to the stem, forming a dome. In some embodiments, the head is resilient. A particular advantage of a resilient dome is that the edges of the dome may bend inward. By bending inward, the effective diameter of the dome is reduced, and a dome which is larger than the vascular access site may be passed into the vein or artery. When the dome is again expanded and deployed, it is larger than the access site and thus may be positioned to occlude the vascular access site.

The stem may be elongated and connected to the dome. An elongated stem is advantageous in that it allows medical personnel to easily control and position the vascular occlusion device manually, and without the use of specialized equipment. Further, by connecting the stem to the dome, the medical personnel may pull on the stem and thus draw the dome against the wall of the blood vessel to improve occlusion.

In one embodiment, the vascular occlusion device is molded from submucosal tissue. Submucosal tissue is resilient and allows the edges of the dome to bend inward, thus facilitating delivery of a larger dome through a smaller puncture. An additional advantage of using submucosal tissue is its biological remodeling attributes. When secured to tissue within a body, submucosal tissue promotes re-growth of endogenous connective tissue to seal the access site. Further, there is a high degree of interspecies similarity among submucosal tissue composition, which also means there is a reduced risk that the vascular occlusion device will be rejected by the host tissue.

In one embodiment, the vascular occlusion device may further comprise a plurality of ribs on the underside of the dome. The ribs are configured to add strength to the dome and to reduce the likelihood that the dome will be inverted during installation and positioning within a vascular access site. The ribs are thus configured to reduce the risk that the dome will invert and be pulled through the puncture in the blood vessel wall. In one embodiment, the ribs extend radially from the stem and terminate before reaching the outer edge of the dome. Partial extension improves strength without adversely affecting occlusion of the vascular access site. With only partial extension, the internal surface of the dome remains smooth adjacent the outer edge of the dome, thus allowing a greater contact area between the dome and the vascular wall.

The vascular occlusion device may further comprise a transition section between the dome and the stem. The transition section may be configured to allow a transition between the dome and stem. In one embodiment, the transition section is tapered such that the diameter is greater nearest the dome and more narrow as the transition section approaches the stem. This tapered section improves the strength of structural strength of the vascular occlusion device by strengthening the connection between the dome and the stem. Additionally, the transition section facilitates manufacture of the vascular occlusion device as well as delivery of the vascular occlusion device to the blood vessel.

In still other embodiments, the vascular occlusion device may comprise a loop in the bottom portion of the stem. Alternatively, the loop may be located in other portions of the vascular occlusion device. The loop is configured to permit the vascular occlusion device to be anchored to the patient. Once anchored to the patient, the vascular occlusion device substantially occludes the vascular access site. When secured to the patient, the occlusion device substantially seals the vascular puncture and the patient may resume a near full range of motion. In one embodiment, the vascular occlusion device is anchored by passing a fiber through the loop and is stitching the fiber to the muscular tissue or skin of the patient.

In one exemplary embodiment, a delivery device is used to deliver the vascular occlusion device to the puncture in the vessel wall. The delivery device is configured to releasably secure the vascular occlusion device to an elongated section of the delivery device. The delivery device may further be configured to selectively position the vascular occlusion device into the blood vessel. The elongated section may comprise an inner lumen and have distal and proximal ends. The distal end is configured to retain the vascular occlusion device and to facilitate bending of at least a portion of the vascular occlusion device. The inner lumen is configured to receive the stem of the vascular occlusion device therein. The inner lumen defines a tapered section that is configured to abut the tapered section of the vascular occlusion device. The tapered section facilitates delivery of the vascular occlusion device by facilitating insertion of the vascular occlusion device into the delivery device, and by transferring the force through the tapered section of the delivery device to the tapered section of the vascular occlusion device.

The present invention also relates to a method for installing a vascular occlusion device to accelerate hemostasis above the natural clotting ability of the body. The method comprises providing an introducer, a delivery device, and a vascular occlusion device. The introducer is inserted into the vascular access site to provide intravascular access. A flexible vascular occlusion device molded of submucosal tissue is introduced into the introducer. The outside diameter of the vascular occlusion device is reduced as the vascular occlusion device is moved toward the distal end of the introducer. The delivery device also passes through the introducer and drives the vascular occlusion device toward the blood vessel.

The invention further relates to a kit configured to promote rapid hemostasis of a vascular access site following a percutaneous, intravascular procedure. The kit includes a vascular occlusion device for occluding the access site, thus blocking the flow of blood from the blood vessel. The kit further includes a delivery device configured to place the vascular occlusion device in a desirable position within a blood vessel. The kit may further comprise an introducer through which the delivery device and the vascular occlusion device pass. The introducer is hollow, which facilitates access to the blood vessel. In other embodiments, the kit may further include a fiber which is used to secure the vascular occlusion device to muscle tissue or skin, or to close the surgical wound. Furthermore, the kit may include other medical components in addition to or instead of the fiber and/or introducer.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A is a frontal view illustrating a vascular occlusion device in accordance with one embodiment of the invention;

FIG. 1B is a bottom view illustrating a vascular occlusion device according to an embodiment of the invention;

FIGS. 2A-2D are cross-sectional views illustrating alternative embodiments of vascular occlusion devices;

FIG. 3A is a cross-sectional view illustrating delivery of a compressed vascular occlusion device according to an embodiment of the invention, following a percutaneous, intravascular procedure;

FIG. 3B is a cross-sectional view illustrating positioning of a vascular occlusion device following a percutaneous, intravascular procedure;

FIG. 4A is a frontal view illustrating an alternative embodiment of a delivery device according to one embodiment of the invention;

FIGS. 4B and 4C are enlarged top and bottom views, respectively, of the delivery device in FIG. 4A;

FIGS. 4D and 4E are cross-sectional views illustrating alternative embodiments of the delivery;

FIG. 5A is a perspective view illustrating a form that is used to make a vascular occlusion device according to one embodiment of the invention;

FIG. 5B is a cross-sectional view illustrating the form of FIG. 5A; and

FIG. 6 is a perspective view illustrating the manufacturing process for making a vascular occlusion device.

### DETAILED DESCRIPTION OF THE PREFERRED EMBODIMENTS

Exemplary embodiments of the present invention relate to a vascular occlusion device configured to promote hemostasis following a percutaneous medical procedure. When properly inserted into a blood vessel, the vascular occlusion device increases the safety of an intravascular procedure by reducing or eliminating leakage from the blood vessel into the surrounding tissue. The vascular occlusion device may be useful in other medical procedures involving other subcutaneous tissue structures which also contain voids. For example, the vascular occlusion device may also be utilized following a procedure involving access to a body cavity or a bodily organ. In one embodiment, the vascular occlusion device is comprised of absorbent biomaterials. Suitable biomaterials include, for example, submucosal tissue or other extracellular matrix-derived tissue which has one or more of several characteristics, including: biological remodeling, resistance to infection, excellent tear resistance and material strength, high similarity to autogenous material minimizes the risk of rejection by the host, and a long shelf life. In addition, the vascular occlusion device can be used with much of the existing medical equipment, thus requiring few customized parts or components for effective use. The vascular occlusion device can be inserted quickly and efficiently, thus reducing the time and complexity of the procedures often necessary to ensure the health and safety of a patent following an intravascular procedure.

Reference will now be made to the drawings to describe various aspects of exemplary embodiments of the invention. It is understood that the drawings are diagrammatic and schematic representations of such exemplary embodiments, and are not limiting of the present invention, nor are they necessarily drawn to scale. No inference should therefore be drawn from the drawings as to the dimensions of any invention or element. In the following description, numerous specific details are set forth in order to provide a thorough understanding of the present invention. It will be obvious, however, to one of ordinary skill in the art that the present invention may be practiced without these specific details. In other instances, well-known aspects of sealing vascular access sites have not been described in particular detail in order to avoid unnecessarily obscuring the present invention.

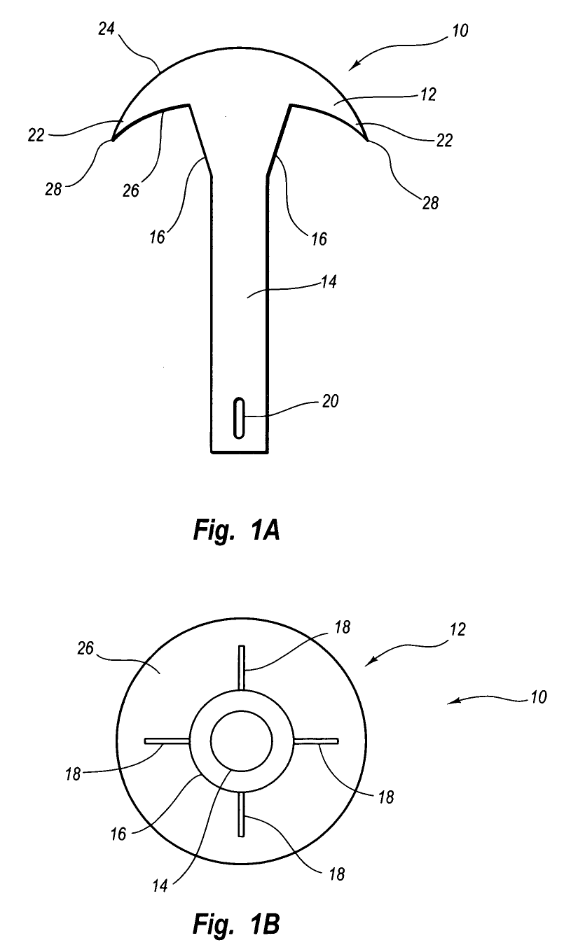

FIGS. 1A and 1B illustrate an exemplary embodiment of a vascular occlusion device 10 according to the present invention. Vascular occlusion device 10 is configured to promote hemostasis. In this embodiment, vascular occlusion device 10 comprises a head 12 connected to a stem 14. The utilization of head 12 and stem 14 provides many advantages. For instance, the use of head 12 with stem 14 allows head 12 to be positioned inside a subcutaneous tissue structure to substantially seal the access site 76, while allowing stem 14 to remain extravascular and extend outside the tissue structure where a medical professional can easily control positioning of vascular occlusion device 10. Additionally, connecting stem 14 to head 12 allows for simplified manufacture of vascular occlusion device 10 as an integral piece, rather than in component parts.

In the illustrated embodiment, head 12 includes an outer surface 24, and ribs 18 located on an inner surface 26 of head 12. Head 12 is generally circular when viewed from the top. Head 12 is configured to accelerate hemostasis and improve occlusion of access site 76. In this embodiment, hemostasis is accelerated and occlusion improved because head 12 has an outside diameter and profile area which is greater than the diameter and profile area of the vascular access site 76. As one of ordinary skill in the art would appreciate, the shape of head 12 may be varied from the illustrated embodiment. For example, head 12 may form an ellipse, which is advantageous if vascular access site 76 is elongated.

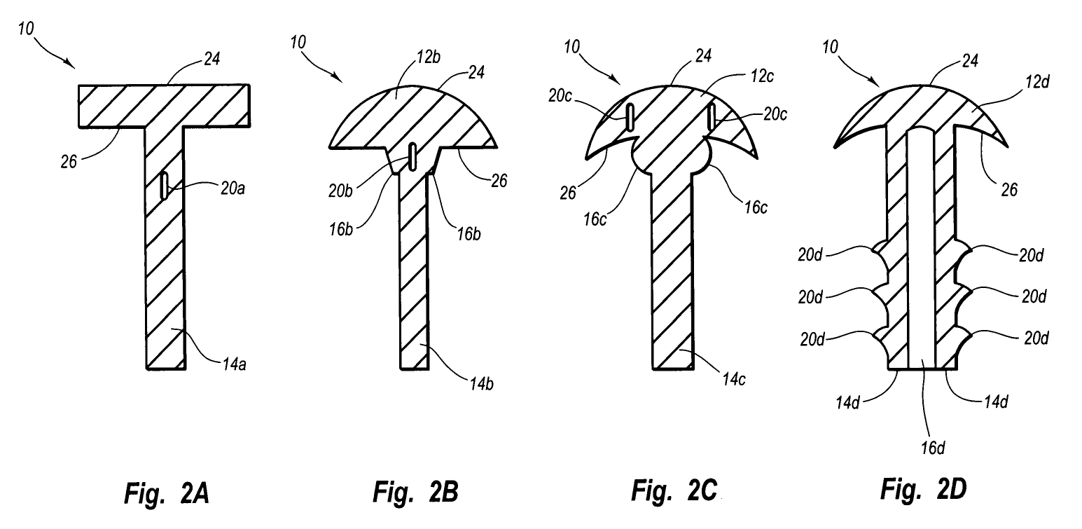

In the illustrated embodiment, outer surface 24 and inner surface 26 are curved and meet at outer edge 28, so as to form a dome which is concave to stem 14. In this manner, head 12 is a circular dome with outer surface 24 forming the exterior dome surface, while inner surface 26 forms the interior dome surface. It is appreciated that alternative embodiments permit other configurations of head 12. For example, in the embodiments illustrated in FIGS. 2A-2B, the outer surface 24 and inner surface 26 may be flat, or only the outer surface 24 may be curved. In still other embodiments, outer surface 24 may not directly contact inner surface 26, and an outer surface may be formed between outer surface 24 and inner surface 26.

In this embodiment, head 12 comprises a pliable material so as to enable head 12 to be flexible. Flexure of head 12 is greatest at the points furthest from the connection to stem 14, including at the wings 22 and at outer edge 28. The flexibility of head 12 provides many advantages. For example, the vascular walls surrounding vascular access site 76 may be uneven. When head 12 is employed to occlude access site 76, wings 22 can flex to accommodate the inconsistencies in order to improve vascular occlusion. In the illustrated example, multiple wings 22 are depicted. Preferably, a single wing 22 is formed as a continuous surface on head 12. Alternatively, wings 22 may be separate such that head 12 comprises a plurality of wings 22.

In one embodiment, vascular occlusion device comprises at least one rib 18. In the illustrated embodiment, ribs 18 are provided on inner surface 26 of head 12. Ribs 18 may be configured to add strength to head 12. More particularly, ribs 18 may be configured to add strength by reducing the flexure of wings 22 and head 12. Additionally, ribs 18 may be configured to add strength by restricting and reducing outward flexure of wings 22, which could result in inversion or facture of head 12. When inverted, the effective diameter of head 12 is reduced such that head 12 may be inadvertently pulled through vascular access site 76 after being positioned to occlude the site. Thus, ribs 18 also improve occlusion of vascular access site 76.

In one embodiment, four ribs 18 begin at the connection of head 12 to stem 14, and extend radially toward outer edge 28. In this embodiment, ribs 18 extend only partially toward outer edge 28. An advantage of ribs 18 being only partially extended toward outer edge 28 is that the inner surface proximate outer edge 28 is substantially smooth. This reduces gaps in the contact area between the area proximate the outer edge 28 and the internal wall of the subcutaneous tissue structure, thus facilitating occlusion of access site 76. In an alternative embodiment, there are no ribs 18, such that the entire inner surface is substantially smooth. It will be appreciated by one of ordinary skill in the art, however, that alternative embodiments allow ribs 18 to extend to the outer edge 28 of head 12. For example, ribs 28 may extend to the outer edge 28 where increased strength of head 12 is desirable or where head 12 is otherwise adapted to improve occlusion, such as where head 12 is flexible. While the embodiment illustrated in FIG. 1B illustrates four ribs 18, it will be appreciated by a person of ordinary skill in the art that a different number of ribs 18 may be provided. Indeed, under some conditions head 12 may comprise no ribs 18 and/or other strengthening elements may be added. For example, one or more tethers between head 12 and stem 14 may add strength. Alternatively, stem 14 may be sufficiently wide to reduce the risk that head 12 will flex outward and invert. Additionally, it is appreciated that while the illustrated ribs 18 formed on the inner surface 26 of head 12 radially extend from the center of head 12, this feature is not limiting. In alternative embodiments it is contemplated that ribs 18 are formed, for example, on the outer surface 24 of head 12. In yet another embodiment, ribs 18 are formed concentrically. In still another embodiment, ribs 18 are formed to produce star patterns.

In the illustrated embodiment, stem 14 is elongated and configured to connect, either directly or indirectly, to head 12. Preferably, stem 14 is substantially solid and cylindrical. However, other cross-sectional shapes are contemplated. For example, it will be appreciated by a person of ordinary skill in the art that cross-section of stem 14 may form an oval, a square, a regular polygon, or a figure eight. Additionally, stem 14 may be hollow, or the thickness of stem 14 may vary along its length.

In one embodiment, stem 14 is configured to be flexible. Like head 12, it is preferred that stem 14 be made of a pliable material to create the flexibility. Flexibility of stem 14 is desirable for many reasons. For example, flexibility in stem 14 increases the resilience of stem 14 and reduces the risk that vascular occlusion device 10 will fracture. Fracture increases the risk that head 12 will be released into an organ, cavity, or blood vessel. A particular risk with intravascular procedures is that head 12 may pass into a smaller vessel where it can contribute to embolism. Additionally, where head 12 is made of a pliable material, stem 14 may be formed integrally with head 12 to reduce manufacturing complexity. Manufacturing complexity is reduced by eliminating a separate component that could otherwise be used to permanently or releasably couple stem 14 to head 12. It will be appreciated by one of ordinary skill in the art that these advantages are representative. For example, in other embodiments, stem 14 may be rigid so as to provide other advantages such as further increased strength and ease of use.

In some embodiments, stem 14 is configured to facilitate retention and reduce the chance that vascular occlusion device 10 will break free and flow into a blood vessel or organ. In the illustrated embodiment, stem 14 includes a loop 20. Preferably, one end of a fiber, such as a suture, is passed through loop 20. By passing the fiber through loop 20, the fiber acts as a safety mechanism, allowing medical personnel to control vascular occlusion device 10 during installation. The fiber and loop 20 further reduce disposal of vascular occlusion device 10 into the subcutaneous tissue structure where it could result in an embolized vein or artery, or contribute to an. infarct. After head 12 is inserted and drawn against the tissue structure, the fiber may be anchored to the tissue surrounding the tissue structure or to the skin to maintain the position of vascular occlusion device 10. Additionally, where the medical procedure creates a lesion in the skin, the fiber may be used for stitches used to suture the lesion. As will be appreciated by a person of ordinary skill in the art, it is not necessary that. loop 20 be formed in the stem for all embodiments of the present invention. In the alternative embodiments illustrated in FIGS. 2A-2D, for example, there may be no. loop, or loop 20 may be formed on head 12 or at other sites on vascular occlusion device 10. In other embodiments, loop 20 may be replaced with barbs 20d or rings configured to anchor the vascular occlusion device 10 to the surrounding tissue.

FIGS. 1A and 1B further illustrate a transition section 16 which is connected to head 12 and stem 14. In this embodiment, transition section 16 is a straight taper which connects to head 12 and narrows as it approaches the connection to stem 14. A transition between stem 14 and head 12 provides many advantages. For example, where head 12 and stem 14 are integrally formed, the manufacturing process is likely to create some taper unless high cost molds or other expensive manufacturing techniques are employed. By including transition section 16 into the design of vascular occlusion device 10, manufacturability is increased while manufacturing costs and complexity are reduced. Additionally, transition section 16 increases the outer diameter in the area adjacent inner surface 26. In this manner, transition section 16 increases the strength of the connection of head 12 to stem 14. The increased strength reduces the flexure of head 12 near the connection to stem 14, such that it reduces the risk that head 12 will invert after deployment into access site 76. This greater strength further improves the ability of vascular occlusion device 10 to occlude access site 76. While the illustrated embodiment provides many advantages, it will be appreciated by one skilled in the art that transition section 16 may be eliminated or modified. In alternative embodiments, such as those illustrated in FIGS. 2A-2D, transition section 16 may, for example, be eliminated, may include shoulders 16b, may comprise a rounded taper 16c, or may be replaced with a hollow section 16d within stem 14.

As disclosed above, in a preferred embodiment of the invention, at least head 12 and stem 14 are made of a pliable material. More preferably, the vascular occlusion device 10 is, formed as an integral apparatus, and all components are made of a pliable material. Many pliable materials may be used to make vascular occlusion device 10. Representative materials include, for example, absorbable biomaterials such as collagen or submucosal tissue or other matrix or scaffolding-based tissue. Preferably, a suitable material will facilitate biological remodeling upon insertion into access site 76 by promoting re-growth of endogenous connective tissues.

Examples of suitable materials include submucosal tissue or other extracellular matrix-derived tissue of a warm-blooded vertebrate including, but not limited to, porcine, ovine, and bovine species. Examples of such suitable submucosal or other extracellular matrix-derived materials are described in U.S. Pat. Nos. 4,902,508, 5,281,422, 5,573,784, 5,573,821, 6,206,931, and 6,790,220, the disclosures of which are herein expressly incorporated by reference. The desired submucosal tissue to be harvested from these warm-blooded vertebrate is acellular and can be used to remodel endogenous tissue. Tissue that remodels endogenous tissue may be found in location such as the intestines, stomach, or urinary bladder. Reference in this application to submucosal tissue is made and is intended to include extracellular matrix. derived tissue or other tissue or material that remodels endogenous tissue. In particular, submucosal tissue refers to naturally derived biomaterials that are biocompatible with a host, and which provide a scaffold for host cells to replace and repair damaged or removed tissue. In an embodiment of the invention, intestinal submucosal tissue is the preferred material used to make vascular occlusion device 10. In a more preferred embodiment, the submucosal tissue is derived from the small intestine of a pig. More particularly still, the submucosal tissue is derived from a layer between the mucosal and muscular layers inside a porcine small intestine. Porcine small intestinal submucosal tissue is advantageous for many reasons. For example, because pigs are raised for meat production, the small intestinal submucosal tissue from a pig is abundantly available as a by-product. It is, therefore, a low cost material.

Forming the vascular occlusion device from submucosal tissue provides many advantages. For example, submucosal tissue has been found to assimilate itself into host tissues, and remodel the implanted environment. The submucosal tissue is biocompatible with the host and does not encapsulate when implanted. In some embodiments, the submucosal tissue comprises a natural structure and composition. Over time, the submucosal tissue is resorbed and replaced with autogenous tissue, such that it develops the features characteristic of the surrounding host tissue. In this manner, the boundaries between the submucosal tissue and the endogenous tissue are substantially imperceptible after repair and remodeling. Additionally, it is well known that submucosal tissue has a high resistance to infection. Submucosal tissue has previously been used in tissue grafts, the majority of which were non-sterile. Despite the large percentage of non-sterile grafts, no complications have arisen due to infection. Preferably, the submucosal tissue is not sterilized, which features a reduction in cost over sterilized submucosal tissue. Optionally, the submucosal material may be sterilized with appropriate methods well known in the art. Exemplary sterilization techniques are discussed in U.S. Pat. No. 6,790,220 which is herein incorporated by reference. Submucosal tissue can be sterilized by gluteraldehde tanning, formaldehyde tanning at an acidic pH, treatment with propylene oxide or ethylene oxide, gas plasma sterilization, gamma radiation, electron beam, or peracetic acid sterilization.

A further advantage of submucosal tissue is that it is readily accepted by the host tissue. Because submucosal tissue exhibits a high degree of interspecies similarity, the host's immune system does not detect the tissue as foreign, and instead adopts the xenogeneic submucosal tissue as its own. Additionally, submucosal tissue exhibits exceptional mechanical properties which include high tear-resistance and high tensile strength. These properties combine to give vascular occlusion device 10 high resistance to fracture, and thus reduce the risk that head 12 will be broken off into the blood vessel or other subcutaneous tissue structure.

Yet another advantage of employing submucosal tissue is its long shelf life. At room temperature and atmospheric pressure, submucosal tissue maintains its condition for more than two months without any substantial loss in performance. When submucosal tissue is dried, either by lyophilization or by air-drying, the shelf life is further increased. Particularly with lyophilized submucosal tissue, the tissue exhibits excellent re-hydration and is re-hydrated quickly once vascular occlusion device 10 is inserted into the blood vessel or other subcutaneous tissue structure, thus facilitating effective occlusion of access site 76.

FIGS. 2A-2D illustrate cross-sectional views of alternative embodiments of vascular occlusion device 10 that are within the scope of the present invention. Different features and characteristics of vascular occlusion device 10 may be necessary for different patients, procedures or subcutaneous tissue structures. The alternative embodiments illustrated in FIGS. 2A-2D are exemplary only, and depict various features and characteristics that may be modified to meet any of various needs. For example, transition section 16 may be eliminated, or it may comprise shoulders 16b or a rounded taper 16c. Alternatively, transition section 16 may comprise a hollow section 16d of stem 14.

In some embodiments, loop 20 may also be modified or eliminated. For example, loop 20 may be removed and replaced with barbs 20d which grab at the surrounding muscle tissue 64 to anchor vascular occlusion device 10. Alternatively, loop 20 may be positioned in head 12, in transition section 16, or at any position along the length of stem 16.

In another example, the configuration of head 12 may vary. Head 12a may be flat on both outer surface 24 and inner surface 26. Alternatively, outer surface 24 may be curved and inner surface 26 may be flat. In yet another embodiment, outer surface 24 may not directly contact inner surface 26. For example, vascular occlusion device 10 may not have outer edge 28 in contact with inner surface 26 and outer surface 24. Instead, in one example, an outer surface may indirectly connect inner surface 26 to outer surface 24.

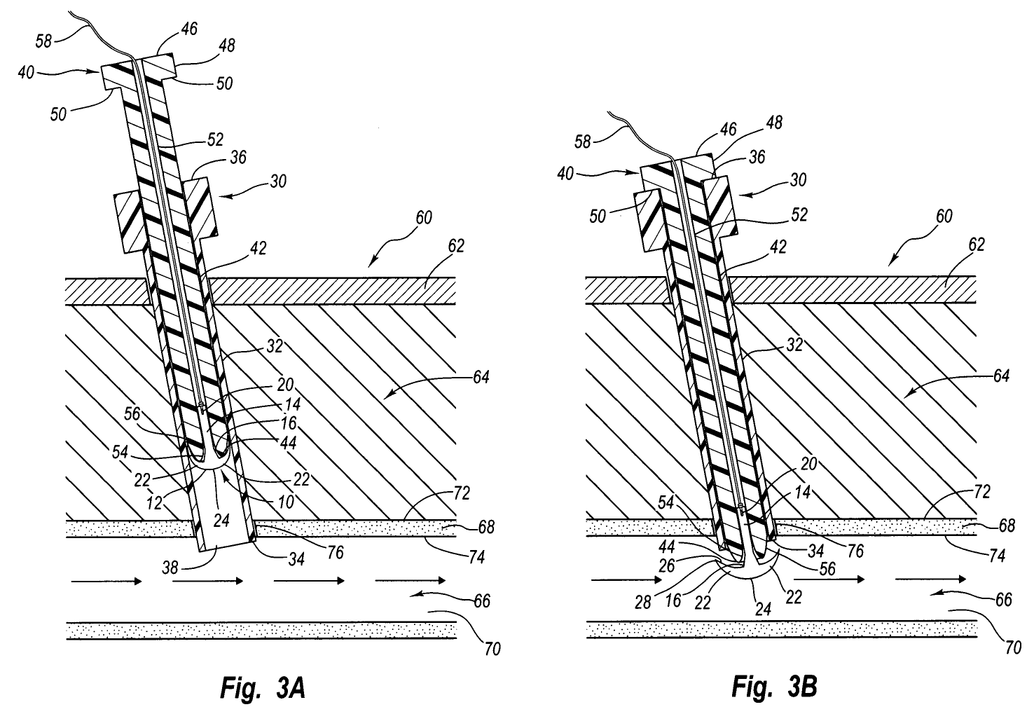

FIGS. 3A and 3B depict cross-sectional views illustrating the positioning of vascular occlusion device 10 within a blood vessel 66. Further illustrated are an introducer 30 and delivery device 40 employed in connection with vascular occlusion device 10. In one embodiment, delivery device 40 is configured to releasably secure vascular occlusion device 10. Delivery device 40 may also be configured to facilitate installation of vascular occlusion device 10 within a subcutaneous tissue structure such as blood vessel 66. Introducer 30 may be configured to provide intravenous access and to facilitate positioning of vascular occlusion device 10. As will be appreciated by one of ordinary skill in the art, vascular occlusion device 10, introducer 30, and delivery device 40 may comprise a kit.

Vascular occlusion device 10 provides rapid hemostasis to arterial or venous vascular access sites 76 following percutaneous procedures. Further, delivery device 40 allows quick and efficient delivery of the vascular occlusion device 10 to the vascular access site 76. Delivery device 40 may be configured for use with a variety of introducers 30. The combination of introducer 30 and delivery device 40 to position vascular occlusion device 10 provides many advantages. For example, this combination provides rapid closure and hemostasis of a vascular access site 76 following a percutaneous procedure. Promotion of hemostasis is accomplished by occluding access site 76 with the vascular occlusion device 10, and allowing for a fiber 58 to be passed through delivery device 40 and introducer 30. Once the vascular occlusion device 10 is deployed, the medical personnel can almost immediately begin suturing the patient to seal the lesion. In this manner, vascular access site 76 is occluded and blood loss is significantly reduced, while the patient may almost immediately regain a near full range of motion.

Additionally, the complexities of promoting hemostasis are reduced. Vascular occlusion device 10 may be used with currently available introducers and equipment used to seal vascular access site 76. This reduces the need for specialized and complicated equipment. For example, delivery device 40 and vascular occlusion device 10 are configured to be used with a variety of introducer models, thus reducing the need to redesign an introducer to accommodate other equipment or components such as delivery device 40. In addition, delivery device 40 and vascular occlusion device 10 can be placed and moved through introducer 30 to the blood vessel 66 without complex procedures to clamp vascular occlusion device 10 to the blood vessel 66 or surrounding tissue 64. Further, additional procedures for positioning vascular occlusion device 10 are reduced and/or eliminated.

In one embodiment, introducer 30 comprises a distal end 34 and a proximal end 36. Additionally, introducer 30 may comprise an elongated section 32, which may further comprise an internal lumen 38. Introducers are commonly used in percutaneous medical procedures. Introducer 30 is exemplary of the type of introducer that may be used in accordance with the present invention. For example, introducer 30 may be adapted for catheterization, or may be needle, sheath, cannula, guide wire, trocar, or any other element used to gain access to a void within a blood vessel, organ, or body cavity.

Introducer 30, as depicted in FIGS. 3A and 3B, may be used when performing catheterization procedures in coronary and peripheral arteries and veins such as the brachial and femoral arteries. For example, a catheter is introduced into the vascular system by first penetrating the skin 62, underlying muscle tissue 64, and the blood vessel 66 with a needle. A guide wire is then inserted through the lumen of the needle and enters blood vessel 66. Subsequently, the needle is removed and an introducer such as introducer 30 is fed over the guide wire and pushed through skin 62. and through vessel wall 68 to enter blood vessel 66. With introducer 30 in place, the guide wire may be removed. Following removal of the guide wire, a catheter is fed through internal lumen 38 of introducer 30 and advanced to a location where the medical procedure or diagnosis is to occur. Well over a dozen medical procedures may be undertaken in this manner, including, for example, angioplasty, stent insertion, PIC line, art line, nephrostomy, and coronary angiography or arteriography. Following any such procedure, the catheter is removed, and access site 76 to blood vessel 66 must be sealed as quickly as possible to avoid complications. Although a typical catheterization procedure is described, the described procedure is exemplary and non-limiting, and the described introducer 30 may be used in any procedure involving access to a subcutaneous tissue structure such as blood vessel 66, a body cavity, or an organ.

Delivery device 40 may comprise a distal end 44, a proximal end 46, and an elongated section 42. In one embodiment, delivery device 40 further comprises a handle 48 at or near proximal end 46. Delivery device 40 may be configured to fit at least partially inside internal lumen 38 of introducer 30. In the illustrated embodiment, elongated section 42 has a generally circular cross section, as does internal lumen 38 of introducer 30, although it will be appreciated that a variety of other cross-sectional shapes may be employed for elongated section 42 and/or internal lumen 38. The diameter of elongated section 42 of delivery device 40 may be configured to be equal to or less than the internal diameter of internal lumen 38 of introducer 30. In such an embodiment, delivery device 40 is allowed to pass through elongated section 32 of introducer 30.

Delivery device 40 may further be configured to facilitate delivery of vascular occlusion device 10 to vascular access site 76. In the illustrated embodiment, delivery device 40 comprises a channel 52. Channel 52 is configured to releasably secure vascular occlusion device 10. In particular, channel 52 is configured to secure stem 14 of vascular occlusion device 10. In some embodiments, the diameter of channel 52 is greater than the diameter of stem 14, thus allowing channel 52 to receive stem 14. In this manner, stem 14 may be placed within channel 52 while vascular occlusion device 10 is passed through introducer 30 and positioned within blood vessel 66. Once positioned, delivery device 40 is retracted and stem 14 is released by channel 52. Preferably, the selective release is performed automatically once the medical personnel retract the delivery. In this embodiment, once head 12 is deployed, the. medical personnel may retract delivery device 30 and stem 14 will be released by. channel 52. As will be appreciated by one having ordinary skill in the art, other embodiments are contemplated. For example, a plunger device (not shown) may extend through channel 52 and push stem 14 of vascular occlusion device 10 to selectively release vascular occlusion device 10.

In an exemplary embodiment, channel 52 has a circular cross-section and is centered within delivery device 40. Channel 52 may be open to ambient only at the distal end 44 and proximal end 46 of delivery device 40. Additionally, channel 52 may have a constant diameter through elongated section 42 and handle 48. It will be appreciated by a person of ordinary skill in the art that other embodiments are within the scope of the present invention. For example, channel 52 may vary in width and position along the length of elongated section 42. Additionally, channel 52 may have any variety of cross-sectional shapes. Further still, channel 52 may be open to ambient along the length or outer surface of elongated section 42.

In one embodiment, channel 52 wholly extends through delivery device 40. An advantage of this feature is that fiber 58 may be connected to loop 20 before insertion of vascular occlusion device 10 into introducer 30, such that fiber 58 may extend through channel 52 of delivery device 40 and be accessible to the medical personnel performing the procedure. In this manner, channel 52 is a safety mechanism which, in conjunction with fiber 58, prevents release of vascular occlusion device 10 into the blood stream. Medical personnel may use the accessibility of fiber 58 to control vascular occlusion device 10, and may extract delivery device 40 and introducer 30 without the danger that occlusion device 10 will be released into the blood flow through blood vessel 66. Further, channel 52 simplifies the installation and positioning of vascular occlusion device 10. Fiber 58 is more easily secured to loop 20 before vascular occlusion device 10 is placed in the body 60, thus speeding the process to occlude access site 76. While in this embodiment it is advantageous that channel 52 extend wholly through delivery device 40, it will be appreciated by one of ordinary skill in the art that for certain applications and with alternative vascular occlusion device 10 configurations, channel 52 may extend through only a portion of delivery device 40, or channel 52 may be eliminated.

Delivery device 40 may further comprise an internal taper 54 at or near distal end 44. Delivery device 40 is configured to facilitate the releasable security of vascular occlusion device 10. Internal taper 54 may be adapted to receive stem 14 and transition section 16 of vascular occlusion device 10. The shape and configuration of internal taper 54 may be such that it substantially matches the shape and contour of the transition section 16 of vascular occlusion device 10 when transition section 16 is received therein. In the illustrated embodiment, transition section 16 comprises a straight taper, and channel 52 similarly defines a straight internal taper 54 of approximately the same angle.

Internal taper 54 provides numerous advantages. For example, internal taper 54 facilitates insertion of stem 14 into channel 52 by providing a wider opening in channel 52 in which to insert stem 14. Additionally, the configuration of internal taper 54 enables a more efficient transfer of force from delivery device 40 to vascular occlusion device 10. The more efficient transfer of force results from an increased contact area between vascular occlusion device 10 and delivery device 40. The efficient transfer of force is desirable because it increases the efficiency of the vascular occlusion device 10, thus permitting the medical personnel to more quickly occlude access site 76 to control bleeding.

Delivery device 40 may further be configured to facilitate compression of vascular occlusion device 10. In the illustrated embodiment, delivery device 40 comprises an external taper 56 on distal end 44. External taper 56 is configured to allow wings 22 of vascular occlusion device 10 to compress towards external taper 56. Additionally, external taper may be configured such that compressed wings 22 will abut external taper 56 without interfering with delivery device 40 when positioned within introducer 30. External taper 56 provides numerous advantages. For example, external taper 56 provides space where wings 22 of vascular occlusion device 10 can bend and be positioned, thus reducing the risk that head 12 will invert. When bent, the effective diameter of head 12 of vascular occlusion device 10 is reduced, thus allowing vascular occlusion device 10 to pass through a smaller introducer 30. This further facilitates positioning of vascular occlusion device 10 into vascular access site 76. Additionally, external taper 56 provides support for the compressed wings 22, thus reducing the risk of fracture in head 12 of vascular occlusion device 10. It will be appreciated by one of ordinary skill in the art that other configurations may be used to facilitate compression of vascular occlusion device 10. For example, an alternative embodiment is contemplated where delivery device 40 facilitates compression of wings 22 by the external diameter of at least a portion of elongated section 42 of delivery, device 40 merely being less than the diameter of internal lumen 38. A reduction of the diameter of elongated section 42, particularly at distal end 44, provides additional space where wings 22 may bend and be positioned, and thus facilitates compression of vascular occlusion device 10.

Delivery device 40 may also be configured to facilitate placement of vascular occlusion device 10 through vascular access site 76. In the illustrated embodiment, delivery device 40 further comprises a handle 48. When pressure is exerted on handle 48, distal end 44 of delivery device 40, and thus vascular occlusion device 10, move toward blood vessel 66 and toward the position illustrated in FIG. 3B. In this embodiment, handle 48 comprises shoulders 50. There are many advantages to delivery device 40 comprising handle 48 and/or shoulders 50. For example, handle 48 may have a larger diameter than elongated section 42, which makes delivery device 40 easier to grip, thus allowing greater precision by medical personnel. Additionally, the length of elongated section 42 may be configured to properly position vascular occlusion device 10. As vascular occlusion device 10 is moved beyond distal end 34 of introducer 30, wings 22 of vascular occlusion device 10 are allowed to expand to their uncompressed orientation. Where the length of elongated section 42 is configured to position vascular occlusion device 10, shoulders 50 contact proximal end 36 of introducer 30 just following extension of wings 22. The contact of shoulders 50 and introducer 30 substantially prevents vascular occlusion device 10 from being inappropriately positioned and pushed completely through blood vessel 66. When contact is made, as illustrated in FIG. 3B, vascular occlusion device 10 is deployed and positioned in blood vessel 66, just beyond distal end 34 of introducer 30. In this manner, vascular occlusion device 10 can be quickly and efficiently placed around vascular access site 76, thus accelerating hemostasis.

In alternative embodiments, delivery device 40 may not have handle 48 and/or shoulders 50. A delivery device 40 without handle 48 may also be advantageous. For example, where handle 48 and shoulders 50 are omitted, introducer 30 may be allowed to pass over delivery device 40. In this manner, introducer 30 may be removed from access site 76 or discarded, while delivery device 40 may remain in place to deploy vascular occlusion device 10. Additionally, delivery device 40 might not be configured such that elongated section 42 can extend only just beyond distal end 34 of introducer 30. For example, as vascular occlusion device 10 passes through internal lumen 38 of introducer 30, wings 22 compress and push against the internal walls of introducer 30. The force against the internal walls of introducer 30 creates resistance to the motion created by pushing on delivery device 40. When vascular occlusion device 10 passes distal end 34 of introducer 30, wings 22 extend and the resistance is reduced or eliminated. The user of delivery device 40 feels the reduction in resistance and understands that vascular occlusion device 10 has been properly positioned. Where the user of delivery device 40 relies on this resistance for appropriately positioning vascular occlusion device 10, it is advantageous that elongated section 42 of delivery device 40 be longer than introducer 30. Such a configuration provides various additional advantages. For example, delivery device 40, does not need to be customized to any particular make or model of introducer 30. Instead, delivery device 40 may be universal and can be used with varying lengths, models, and makes of introducers.

In one embodiment, delivery device 40 is a dilator. Dilators are available in a variety of sizes, shapes, and configurations. For example, dilators are available that comprise a flexible polymer such as polyether urethane. Where such a dilator is used as a delivery device, the dilator may flex as pressure is exerted to move vascular occlusion device 10 down internal lumen 38 of introducer 30. In alternative embodiments, it is advantageous that elongated section 42 of delivery device 40 be stiff, rigid, or otherwise resistant to flexure. For example, where delivery device 40 must pass through a hemostasis valve on introducer 30, a stiff elongated section 42 allows more control by the medical personnel and less effort need be exerted by the user. Additionally, if wings 22 create a strong resistance to movement through introducer 30, a flexible elongated section 42 may bend and contact the internal walls of introducer 30. When delivery device 40 bends to contact the internal walls of introducer 30, delivery device 40 transfers some force to introducer 30 rather than to vascular occlusion device 10. Accordingly, a stiff elongated section 42 more effectively distributes the force to vascular occlusion device 10 by avoiding at least some distribution to introducer 30. In alternative embodiments of a flex-resistant delivery device 40, elongated section 42 may comprise a metal, composite, polymer, or other material that is resistant to flexure. Representative materials suitable to form elongate section 42 include, without limitation, stainless steel, polycarbonate, polypropylene, polyamides, and reinforced polyethylene terephthalate.

FIGS. 3A and 3B further depict vascular occlusion device 10 as it is utilized to accelerate hemostasis. Head 12 of vascular occlusion device 10 may be configured to occlude vascular access site 76. In one embodiment, the diameter of head 12 is greater than the width or diameter of vascular access site 76, and thus the profile area of head 12 is larger than the profile area of access site 76. Vascular occlusion device 10 may comprise a pliable material which allows wings 22 to flex. In tandem with the delivery device 40, vascular occlusion device 10 is inserted into introducer 30, thus causing wings 22 on head 12 to compress. Upon traversing the length of introducer 30, wings 22 expand and deploy, thus giving head 12 the maximum diameter and profile area. Vascular occlusion device 10 is thus configured to improve occlusion of access site 76 by providing a greater coverage area to fully cover and occlude vascular access site 76.

Vascular occlusion device 10 may further comprise loop 20 as an exemplary retention device. When installing vascular occlusion device 10, a fiber 58 may be passed through loop 20, and vascular occlusion device 10 may be secured to surrounding muscle tissue 64 or skin 62 upon removal of introducer 30. In a preferred embodiment, loop 20 is located in stem 14. More preferably, loop 20 is located, with reference to the head 12, in the lower third of stem 14. This reduces the interference by fiber 58 in the positioning of head 12 to occlude access site 76.

In alternative embodiments, loop 20 is provided in other areas of vascular occlusion device 10. For example, loop 20 may be located in transition section 16 or an upper or middle third of stem 14. A particular advantage of locating loop 20 more proximate head 12 is that stem 12 does not need to be configured to be any particular length. If stem 14 is too long, it may be trimmed without damaging loop 20. In contrast, where stem 14 is too long and loop 20 is located near the end of stem 14, the medical personnel may not be able to trim stem 14 without damaging loop 20. If stem 14 is too long and cannot be trimmed, medical personnel may have to repeat the procedure with a vascular occlusion device 10 having a shorter stem 14.

Vascular occlusion device 10 may be made in varying sizes to accommodate different procedures, patients, and blood vessels. For example, a larger head is preferably used to occlude an access site when a larger french size introducer is used. Generally, the larger the introducer, the larger the profile area of the access site. While a person of ordinary skill in the art will appreciate that various head sizes may be used for any introducer size, it is preferred that the french size of the head of a vascular occlusion device be at least twice the french size of the introducer 30. This relationship between the size of the head and the introducer provides various advantages. For example, a head that is twice as large as the introducer typically provides sufficient coverage to suitably cover and occlude the access site, which may be about the same size as the external diameter of an introducer. Additionally, a two-to-one relationship balances the effectiveness of the vascular occlusion device by providing a sufficiently large profile area while also providing sufficient resistance inside the introducer to allow effective placement of the vascular occlusion device.

The length and width of the stem 14 may also vary. In one embodiment, the length of stem 14 is configured to reduce infection of the lesion. The end of stem 14 opposite head 12 may be positioned within muscle tissue 64 and terminate before skin 62, while head 12 is positioned inside blood vessel 66. A feature of this configuration is decreased risk of infection. Where stem 14 does not extend through skin 62, the lesion may be sutured and completely sealed to ward off infection. In contrast, where stem 14 extends through skin 62, there may be an increased risk that stem 14 will serve as a conduit for infection, and that infection may ultimately reach the blood stream. However, it is contemplated in other embodiments that stem 14 may extend through skin 62. These alternative embodiments are particularly effective where stem 14 is secured to skin 14 after vascular occlusion device 10 is positioned. Additionally, vascular occlusion device 10 may comprise a sterilized material to reduce the risk of infection. The length of stem 14 may thus vary based on the depth of muscular tissue 64 and skin 62, the procedure to be performed, and the preference of the medical personnel.

In one embodiment, the width of stem 14 is such that the french size of head 12 is between about two and four times larger than that of stem 14. In one respect, a larger stem width contributes to a stronger vascular occlusion device 10 by strengthening the connection between head 12 and stem 14. However, a wider stem 14 also reduces size of wings 22, thus resulting in less surface area to press against a vessel wall 68 and occlude blood vessel 66. A width of stem 12 that is between about two and four french sizes smaller than the french size of head 12 balance these considerations and provides sufficient strength while also allowing wings 22 to flex and head 12 to occlude access site 68.

In accordance with the present invention, a kit is provided for sealing access site 76 in a subcutaneous tissue structure such as blood vessel 66. In one embodiment, the kit includes at least vascular occlusion device 10 and delivery device 40. In another embodiment, an introducer 30 is included as an additional medical component. In yet another embodiment, a fiber 58 is included in the kit. As will be appreciated by one having ordinary skill in the art, introducer 30 and fiber 58 are exemplary only, and other medical components may be included in the kit. For example, one or more introducers or fibers may be provided. A representative list of other medical components includes, but is not limited to: needles, catheters, guide wires, dye, stents, sensors, and balloons.

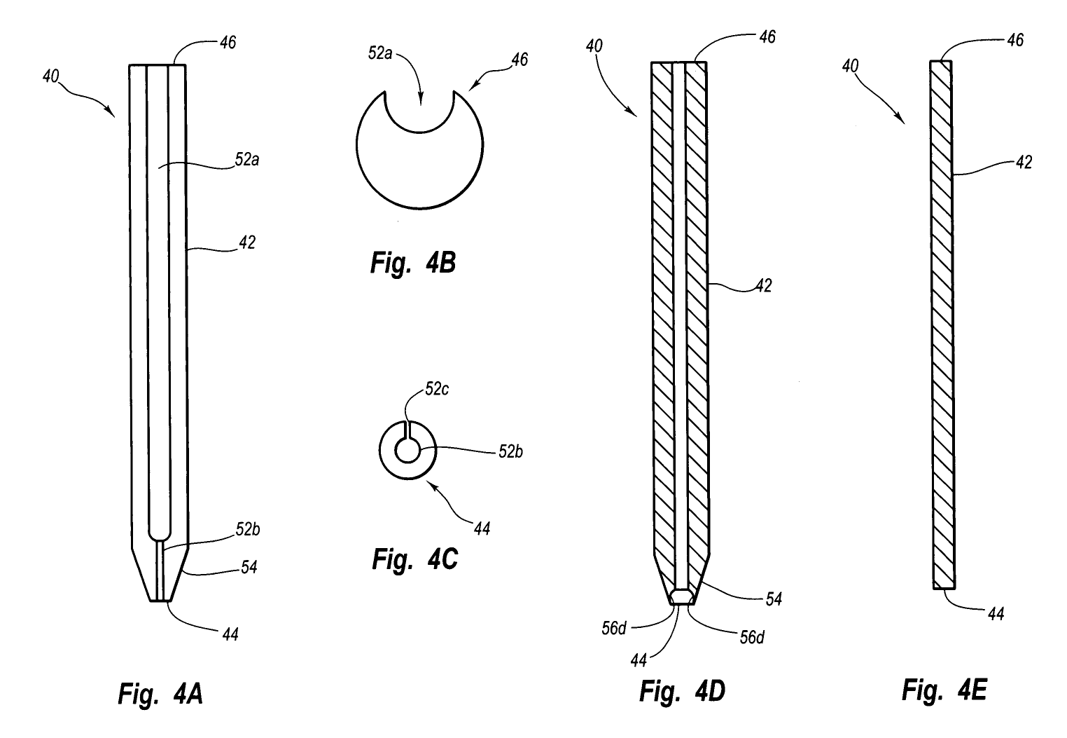

FIGS. 4A to 4C illustrate exemplary, alternative embodiments of delivery device 40. In the illustrated embodiments, delivery device 40 does not include a handle. In some embodiments, delivery device 40 comprises elongated section 42 with distal end 44 and proximal end 46. Elongate section 42 may further comprise proximal channel 52a and distal channel 52b. Proximal channel 52a may be in communication with an outer surface of elongated section 42, such that proximal channel 52a is open to ambient. This configuration is advantageous for various reasons. For example, as noted previously, delivery device 40 may be stiff. A flexible delivery device 40 may be easily extruded from a pliable material. However, where delivery device 40 is stiff, it is difficult to mold or extrude elongated section 42 of suitable dimensions, particularly if a portion of elongated section 42 is hollow. In one embodiment, delivery device 40 is generally solid, and proximal channel 52a is molded, milled, or otherwise formed in elongated section 42. Channel 52 may further be in communication with the outer surface of elongated section 42. In this manner, manufacturing complexity and cost are decreased. In an exemplary embodiment, proximal channel 52a has a semi-circular cross section. As will be appreciated by one having ordinary skill in the art, a variety of shapes and configurations of proximal channel 52a are within the scope of the present invention.

Distal end 44 of elongate section 42 may comprise distal channel 52b. In one embodiment, distal channel 52b is centered within distal end 44, and distal channel 52b is configured to be in communication with proximal channel 52a. In this manner, a fiber 58 may pass between distal channel 52b and proximal channel 52a, and thus on to the medical personnel using delivery device 40. The cross-sectional shape of distal channel 52b may also be generally circular, although other shapes are contemplated. Distal channel 52b is advantageous for many reasons. For example, distal channel 52b may be configured to releasably secure stem 14. The diameter of distal channel 52b may be greater than the diameter of stem 14. In this manner, delivery device 40 can receive stem 14 while vascular occlusion device 10 is pushed through introducer 30 and positioned into a subcutaneous tissue structure. Upon positioning vascular occlusion device 10, delivery device 40 can be retracted and may then release vascular occlusion device 10. Additionally, distal channel 52b may be molded, drilled, or milled into delivery device 40.

In one embodiment, distal channel 52b comprises a slit 52c which is in communication with the outer surface of elongated section 42 and with distal channel 52b. Slit 52c may vary in depth. Preferably, distal channel 52b is centered in distal end 44, and slit 52c is sufficiently deep to be in communication with distal channel 52b. However, it is not necessary that distal channel 52b be centered, and the depth of slit 52c may vary according to the position of distal channel 52b. Through slit 52c, distal channel 52b may be open to ambient. Slit 52c is advantageous for various reasons. For example, slit 52c may be configured to allow fiber 58 to enter distal channel 52b of delivery device 40. It will be appreciated; however, that slit 52c is not necessary in all embodiments of the present invention. For example, in one embodiment, distal channel 52b may be wholly within elongated section 42 of delivery device 40, and not have access to ambient except through distal end 44 and/or proximal channel 52a.

FIGS. 4D and 4E are cross-sectional views illustrating additional exemplary embodiments of a delivery device 40 that are within the scope of the present invention. Delivery device 40 may be configured to releasably secure vascular. occlusion device 10, and to push vascular occlusion device 10 through introducer 30 into vascular access site 76. In one embodiment, delivery device 40 comprises a rounded taper 56d on distal end 44. Rounded taper 56d may be configured to match a corresponding transition section 16c of vascular occlusion device 10, such as that illustrated in FIG. 2C. Alternatively, delivery device 40 may be configured to fit within a hollow transition section 16d of stem 14, such as that illustrated in FIG. 2D. In one embodiment, the outer diameter of at least a portion of elongated section 42 is less than the internal diameter transition section 16 of stem 14. It will be appreciated that these embodiments are exemplary only, and other embodiments are within the scope of the present invention. For example, the width of elongated section 42 may vary, or a handle may be provided.

Vascular occlusion device 10 according to the present invention may be manufactured in any conventional manner known in the art. However, vascular occlusion devices comprising submucosal tissue have previously been limited to simple, conventional shapes such as sheets, cylinders, rolls, and disks. Accordingly, there is a need for a manufacturing process that allows submucosal tissue to be formed into non-conventional shapes, or complex shapes comprising more than one conventional shape.

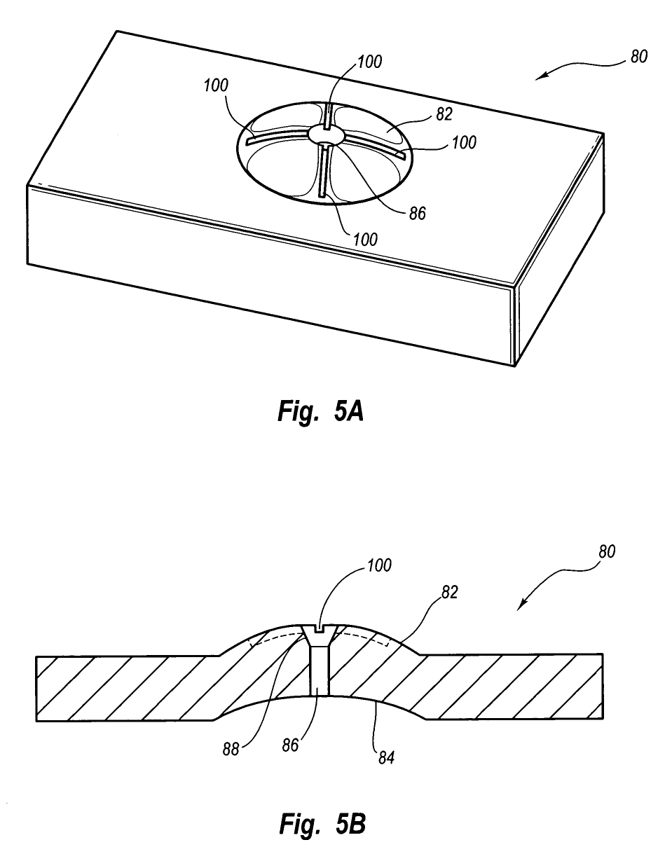

FIGS. 5A and 5B depict perspective and cross sectional views of a form 80 which is configured to mold submucosal tissue into a complex shape, such as vascular occlusion device 10. In one embodiment, form 80 is configured to form a vascular occlusion device 10 which comprises head 12, ribs 18, and stem 14. Form 80 may be formed out of Teflon or another suitable material. Form 80 may be configured to mold a vascular occlusion device 10 with a circular head 12. In one embodiment, form 80 comprises a raised upper portion 82 configured to define the contour of head 12. Form 80 may also comprise a raised lower portion 84. Upper portion 82 may further comprise crevices 100 which may be configured to define the size and position of ribs 18 on vascular occlusion device 10. In the illustrated form 80, four ribs 18 will be created by the four crevices 100. An orifice 86 may also be provided approximately in the center of raised upper portion 82 and may extend through form 80 to raised lower portion 84. In one embodiment, orifice 86 comprises a tapered portion 88 to form transitional section 16 of vascular occlusion device 10.

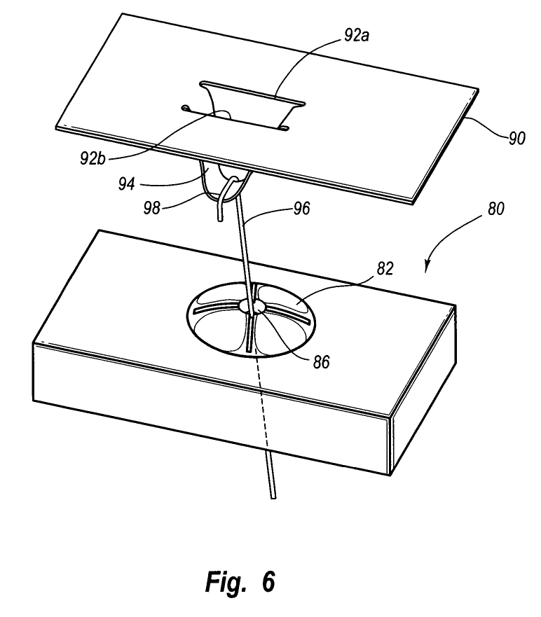

FIG. 6 depicts a manufacturing process to manufacture vascular occlusion device 10 utilizing form 80. Vascular occlusion device is manufactured by providing a sheet 90 of submucosal tissue. In one embodiment, sheet 90 is at least as large as raised upper portion 82 of form 80, and preferably at least as large as the top surface of form 80. Sheet 90 may comprise slits 92a, b which are generally parallel and substantially equal in length. Slits 92a, b may be cut into sheet 90 with, for example, a knife or scalpel. Preferably, slits 92a, b are cut approximately in the middle of sheet 90, and form middle section 94.

athering tool 96 may further be provided. Preferably, gathering tool 96 comprises a hook 98. Hook 98 may be inserted into slits 92a, b and can gather middle section 94 of sheet 90 as shown. Gathering tool 96 may then be inserted through orifice 86, and pulled entirely through form 80 as sheet 90 is positioned on form 80. In pulling gathering tool 96 through orifice 86, middle section 94 is also pulled therethrough. Gathering tool 96 may be removed and set aside once it has pulled middle section 94 through form 80. Once pulled through form 80, middle section 94 may be twisted to form a generally cylindrical stem. However, in one embodiment, a portion of middle section 94 remains untwisted so as to form an opening or loop within the stem.

The portion of sheet 90 which did not pass though orifice 86 remains on the top surface of form 80. This portion of sheet 90 is smoothed against form 80 to substantially eliminate any air bubbles between sheet 90 and form 80. If desirable, additional layers of submucosal tissue may be provided. Preferably, the additional layers are at least as large as form 80, or substantially the same size as sheet 90. Each additional layer may be placed upon the top layer of tissue and compressed and smoothed to eliminate air bubbles between the layers. Preferably, two or three layers of submucosal tissue are used.

In alternative embodiments, additional layers are first placed directly on the top surface of form 80 and compressed and smoothed to eliminate or reduce air bubbles between the layers. Holes approximately the same size as orifice 86 may then be cut in the additional layers above orifice 86. Sheet 90 may then be applied as before, with gathering tool 96 and middle section 94 passing through the additional layers and form 80. Sheet 90 rests on the additional layers and is then compressed and smoothed to reduce air bubbles. A particular advantage of this alternative manufacturing process is a reduced risk of delamination during utilization of vascular occlusion device 10. Because sheet 90 passes through the additional layers, thus forming the stem and the upper-most layer, all layers are compressed together as vascular occlusion device 10 is deployed and positioned in vascular access site 76, and while secured in place by stem 14.

Once all layers are compressed and smoothed, form 80 and the associated layers may be dried. In some embodiments, vascular occlusion device 10 is dried and re-hydrated either just before use or during use in occluding an access site 76. Drying may be accomplished by any conventional method. For example, form 80, sheet 90, and any additional layers may be lyophilized or air-dried. Different benefits may be obtained based on whether the vascular occlusion device 10 is lyophilized or air-dried. For example, lyophilized vascular occlusion devices are less rigid and re-hydrate more quickly when introduced into the blood vessel, organ, or body cavity, thus providing more rapid hemostasis. In contrast, air-drying may be accomplished more quickly, which results in a shortened manufacturing cycle and reduced manufacturing costs. Additionally, air-dried submucosal tissue is more dense and stiff than lyophilized submucosal tissue. This increased stiffness reduces the chance that head 12 will invert either while vascular occlusion device 10 is extended through introducer 30 or is deployed in a subcutaneous tissue structure. Inversion is particularly undesirable once vascular occlusion device 10 is inside the blood vessel because it reduces the effective outer diameter and the profile area of vascular occlusion device 10. In this manner, inversion may allow vascular occlusion device 10 to be pulled through the vascular access site. If inversion occurs during installation of the vascular occlusion device 10, it may be necessary to repeat the procedure. If inversion occurs after the procedure is completed and the lesion is sealed, the patient's safety is compromised if vascular occlusion device 10 passes into the surrounding tissue, because bleeding may again result.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. An occlusion device for substantially sealing an access site to a subcutaneous tissue structure, comprising;
a flexible head; and
a stem coupled to the head, wherein the stem is configured to facilitate positioning of the head when the head is within a subcutaneous tissue structure; and
wherein the head and stem comprise submucosal tissue.

2. The occlusion device as recited in claim 1, wherein the head forms a dome with at least one wing, wherein the wing is compressible, enabling the head to be received within an access site of the subcutaneous tissue structure.

3. The occlusion device as recited in claim 2, wherein the dome is concave.

4. The occlusion device as recited in claim 1, wherein the head comprises at least one radially extending rib configured to reduce flexure of the head in at least one direction.

5. The occlusion device of claim 4, wherein the head comprises a first surface and a second surface, each surface being concave, and wherein the at least one radially extending rib is configured to reduce flexure of the second surface in a direction toward the first surface.

6. The occlusion device as recited in claim 5, wherein the at least one radially extending rib is formed on the second surface, the second surface being on an underside of the head.

7. The occlusion device as recited in claim 4, wherein the at least one radially extending rib is configured to reduce flexure of the head in a direction opposite the stem when the head is within a subcutaneous tissue structure.

8. The occlusion device as recited in claim 1, further comprising a transition section coupling the head to the stem, wherein the transition section is adapted to receive a force to facilitate movement of the head into the subcutaneous tissue structure.

9. The occlusion device as recited in claim 8, wherein the transition section is tapered.

10. The occlusion device as recited in claim 9, wherein the taper increases the surface area over which the force is applied.

11. An occlusion device as recited in claim 1, wherein the stem comprises a loop.

12. The occlusion device as recited in claim 11, wherein the loop is configured to receive a suture therethrough, the suture being utilized to secure the occlusion device.

13. An occlusion device as recited in claim 1, wherein the head is at least twice as large as the access site.

14. An elongated device for percutaneous delivery of an occlusion device into a subcutaneous tissue structure to substantially seal an access site in the subcutaneous tissue structure, comprising:
an elongated section having a proximal end and a distal end, wherein the distal end is configured to receive and substantially retain therein a stem of an occlusion device, and wherein an outer surface adjacent the distal end of the elongated section is configured to facilitate bending of a head of the occlusion device toward the proximal end.

15. An elongated device as recited in claim 14, wherein the distal end is further configured to support at least one resilient wing of the head, where the head bends toward the proximal end of the elongated device.

16. An elongated device as recited in claim 14, wherein the distal end comprises an external taper configured to facilitate bending of the head of the occlusion device.

17. An elongated device as recited in claim 14, wherein an inner surface adjacent the distal end of the elongated section is configured to increase the surface area of the elongated device in contact with the occlusion device, and wherein a force is transferred from the delivery device to the occlusion.

18. An elongated device as recited in claim 14, wherein the elongated section comprises a first channel.

19. An elongated device as recited in claim 18, wherein the first channel is in communication with the outer surface of the elongated section.

20. An elongated device as recited in claim 18, wherein the distal end comprises a second channel configured to receive the stem of the occlusion device, and wherein the second channel is in communication with the first channel.

21. A method for sealing an access site in a subcutaneous tissue structure, the method comprising:
inserting an introducer element into an access site of a subcutaneous tissue structure, wherein the introducer element is configured to provide access to the access site, and wherein the introducer element comprises an inner lumen;
compressing an occlusion device having a flexible head that is larger than the inner lumen of the introducer element and a stem coupled to the head to facilitate securement of the occlusion device, the head and stem comprising submucosal tissue, wherein the head of the occlusion device is compressed enabling the introducer element to receive the occlusion device;
inserting the head into the access site; and
securing the occlusion device to substantially seal the access site.

22. The method as recited in claim 21, further comprising: inserting a delivery device into the introducer, wherein the delivery device is configured to receive the stem of the occlusion device therein.

23. The method as recited in claim 21, further comprising the step of releasably securing the delivery device to the resilient sealing member, wherein the delivery device comprises a distal end configured to receive the sealing member.

24. The method as recited in claim 23, wherein the distal end is adapted to facilitate compression of the sealing member.

25. A kit for sealing an access site to a subcutaneous tissue structure, the kit comprising:
an occlusion device comprising a stem and a head connected to the stem, wherein the stem is configured to facilitate securement of the head against a wall of a subcutaneous tissue structure when the head is positioned within the subcutaneous tissue structure, and wherein the head comprises at least one flexible wing such that the head can be compressed and received within an access site having an profile area less than the profile area of the head, the occlusion device further comprising submucosal tissue; and
a delivery device configured to receive the occlusion device therein and position the occlusion device within the access site, the delivery device comprising a distal end configured to support the at least one flexible wing of the occlusion device and to facilitate compression of the head.
